(19) United States
(12) Patent Application Publication
(10) Pub. No.: US 2024/0174633 A1
(43) Pub. Date: May 30, 2024

(54) CRYSTALLINE FORMS OF (4S)-24-CHLORO-4-ETHYL-73-FLUORO-35-METHOXY-32,5-DIOXO-14-(TRIFLUORO-METHYL)-32H-6-AZA-3(4,1)-PYRIDINA-1(1)-[1,2,3]TRIAZOLA-2(1,2),7(1)-DIBENZENAHEPTAPHANE-74-CARBOXAMIDE

(71) Applicant: Bayer Aktiengesellschaft, Leverkusen (DE)

(72) Inventors: Guillaume LEVILAIN, Wuppertal (DE); Tia JACOBS, Wuppertal (DE); Britta OLENIK, Bottrop (DE); Franco RUBINO, Wuppertal (DE); Krischan ZIEM, Wuppertal (DE); Michal SOWA, Wuppertal (DE); Susanne RÖHRIG, Hilden (DE)

(21) Appl. No.: 18/549,281

(22) PCT Filed: Mar. 4, 2022
(86) PCT No.: PCT/EP2022/055521

§ 371 Date: Sep. 6, 2023

(30) Foreign Application Priority Data
Mar. 9, 2021 (EP) 21161489.6

(51) Int. Cl. C07D 401/10

(52) U.S. Cl. CPC C07D 401/10

(57) ABSTRACT

The present invention relates to crystalline forms of (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)-dibenzenaheptaphane-7⁴-carboxamide which are the crystalline modification I and the crystalline modification II, to processes for their preparation, to pharmaceutical compositions comprising them and to their use in the control of disorders.

The present invention relates to crystalline forms of (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2), 7(1)- dibenzenaheptaphane-7⁴-carboxamide which are the crystalline modification I and the crystalline modification II, to processes for their preparation, to pharmaceutical compositions comprising them and to their use in the control of disorders.

Compound of the formula (I), (4S)-2⁴-chloro-4-ethyl-7³-fluoro-3⁵-methoxy-3²,5-dioxo-1⁴-trifluoromethyl)-3²H-6-aza-3(4,1)-pyridina-1(1)-[1,2,3]triazola-2(1,2),7(1)-dibenzenaheptaphane-7⁴-carboxamide, also named as 4-({(2S)-2-[4-{5-chloro-2-[4-(trifluoromethyl)-1H-1,2,3-triazol-1-yl]phenyl}-5-methoxy-2-oxopyridin-1(2H)-yl]butanoyl}amino)-2-fluorobenzamide, is known from WO2017/005725 and has the following formula:

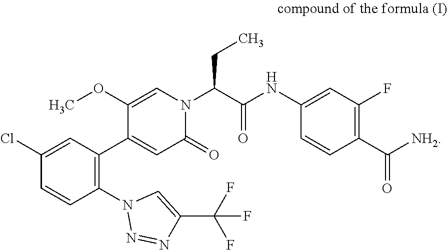 [SMILES: CC[C@@H](C(=O)Nc1ccc(C(N)=O)c(F)c1)n1cc(OC)c(-c2cc(Cl)ccc2-n2cc(C(F)(F)F)nn2)cc1=O]

The compound of the formula (I) acts as a factor XIa inhibitor and, owing to this specific mechanism of action, is, after oral administration, useful in the treatment and/or prophylaxis of disorders, preferably thrombotic or thromboembolic disorders and/or thrombotic or thromboembolic complications, in particular cardiovascular disorders including coronary artery disease, angina pectoris, myocardial infarction or stent thrombosis, as well as disorders in the cerebrovascular arteries and other disorders, leading to transitory ischaemic attacks (TIA), ischemic strokes including cardioembolic as well as non-cardioembolic strokes, and/or disorders of peripheral arteries, leading to peripheral artery disease, including peripheral artery occlusion, acute limb ischemia, amputation, reocclusions and restenoses after interventions such as angioplasty, stent implantation or surgery and bypass, and/or stent thrombosis.

The compound of the formula (I) can be prepared as described in WO2017/005725 in Example 234 and Example 235. Using the described process the compound of the formula (I) is obtained in the amorphous form. The obtained compound of the formula (I) in amorphous form could not be transformed to a crystalline solvent-free form, even by conducting numerous experiments, such as e.g. 1) dissolving the compound of the formula (I) in a solvent and performing typical crystallization experiments including e.g. evaporation of the solvent and cooling of the solutions, or 2) slurrying saturated solutions of the compound of the formula (I) in amorphous form. Different types of solvents as well as mixtures of solvents have been tried.

In WO2019/175043 it is described that the compound of the formula (I) cannot be isolated in a crystalline solvent-free form, but the compound of the formula (I) contained in a racemic mixture does crystallize. This behavior for crystallization of the compound of the formula (I) contained in a racemic mixture is used to produce in an easy and scalable way the compound of the formula (I) (enantiomerically pure) in an amorphous solid state form. The racemic material containing the compound of the formula (I) is crystalline with much lower solubility in organic solvents. Based on this principle of different kinetic solubilities of the desired compound of the formula (I) (enantiomerically pure) in amorphous form and the racemic material containing the compound of the formula (I) in crystalline form, the compound of the formula (I) (enantiomerically pure) is obtained with high ee-values.

The aim of the development was, therefore, to provide the compound of the formula (I) in a crystalline solvent-free form.

Surprisingly, it was found that the compound of the formula (I) in the amorphous form can be dissolved in a solvent and after seeding with a compound of the formula (II) in the crystalline modification A the compound of the formula (I) does crystallise in the crystalline modification I.

The amorphous form can be characterised by an X-ray powder diffractogram displaying no characteristic reflections, as well as a DSC thermogram displaying no melting events (FIGS. 17 and 16). It has now been found that the amorphous form shows hygroscopicity and less stability in comparison to the crystalline modification I.

The following crystalline forms of the compound of the formula (I) have been identified which are the crystalline modification I and the crystalline modification II. In the context of the present invention modifications, polymorphic forms and polymorphs have the same meaning. These crystalline forms exist in addition to the amorphous form. All together—the crystalline forms and the amorphous form—are different solid forms of the compound of the formula (I).

The crystalline modification I of the compound of the formula (I) shows beneficial properties over the amorphous form of the compound of the formula (I) with regard to hygroscopicity and thermal stability. The dynamic vapour sorption isotherms of the amorphous form, the crystalline modification I and the crystalline modification II show that at 80% relative humidity the samples gained 3.2%, 0.04% and 2.13% mass of water respectively. Thermal stability was investigated by storing samples in closed containers for 1 week at 90° C., then measuring the sum of all organic impurities with HPLC (Method 3). 4.4% of organic impurities was measured for the amorphous form, whereas no organic impurities were detected for the crystalline modification I after storage.

Crystalline modification I of the compound of the formula (I) is the thermodynamically stable form below the melting point.

The crystalline modification I of the compound of the formula (I) is therefore suitable for use in the pharmaceutical field, in particular suitable for pharmaceutical compositions.

A pharmaceutical composition according to the present invention comprises the crystalline modification I of the compound of the formula (I) and optionally further pharmaceutically acceptable excipients.

The different forms of the compound of the formula (I) can be distinguished by X-ray powder diffraction, differential scanning calorimetry (DSC), IR- and Raman-spectroscopy.

The crystalline modification I of the compound of the formula (I) can be characterized by infrared spectroscopy which displays at least the following values of the band maxima (cm⁻¹): 1705, 1641, 1429, preferably at least the following values of the band maxima (cm⁻¹): 1705, 1641, 1503, 1429, 791, more preferably at least the following values of the band maxima (cm⁻¹): 1705, 1641, 1503, 1429, 1383, 1039, 791, most preferably at least the following values of the band maxima (cm⁻¹): 3401, 1705, 1613, 1641, 1503, 1429, 1383, 1205, 1039, and 791. The compound of the formula (I) in the crystalline modification I can also be characterized by IR spectrum as shown in FIG. 7.

The crystalline modification II of the compound of the formula (I) can be characterized by infrared spectroscopy which displays at least the following values of the band maxima (cm⁻¹): 1664, 1571, 1134, preferably at least the following values of the band maxima (cm⁻¹): 1664, 1571, 1525, 1373, 1134, more preferably at least the following values of the band maxima (cm⁻¹): 1664, 1571, 1525, 1417, 1373, 1134, 1032, most preferably at least the following values of the band maxima (cm⁻¹): 1664, 1571, 1525, 1417, 1373, 1134, 1032, 870, 825 and 775. The compound of the formula (I) in the crystalline modification II can also be characterized by IR spectrum as shown in FIG. 8.

The crystalline modification I of the compound of the formula (I) can be characterized by Raman spectroscopy which displays at least the following values of the band maxima (cm⁻¹): 1625, 1239, 991, preferably at least the following values of the band maxima (cm⁻¹): 1625, 1572, 1528, 1239, 991, more preferably at least the following values of the band maxima (cm⁻¹): 1625, 1572, 1528, 1359, 1329, 1239, 991, most preferably at least the following values of the band maxima (cm⁻¹): 3059, 1694, 1625, 1572, 1528, 1431, 1359, 1329, 1239 and 991. The compound of the formula (I) in the crystalline modification I can also be characterized by Raman spectrum as shown in FIG. 9.

The crystalline modification II of the compound of the formula (I) can be characterized by Raman spectroscopy which displays at least the following values of the band maxima (cm⁻¹): 1623, 1604, 1336, preferably at least the following values of the band maxima (cm⁻¹): 1623, 1604, 1527, 1336, 981, more preferably at least the following values of the band maxima (cm⁻¹): 1663, 1623, 1604, 1527, 1247, 1336, 981. most preferably at least the following values of the band maxima (cm⁻¹): 1710, 1663, 1623, 1604, 1527, 1374, 1247, 1336, 981 and 709. The compound of the formula (I) in the crystalline modification II can also be characterized by Raman spectrum as shown in FIG. 10.

The crystalline modification I of the compound of the formula (I) can be characterized by a X-Ray powder diffractogram (at 20±5° C. and with Cu—K alpha 1 as radiation) which displays at least the following reflections: 17.8, 19.1, 25.5, preferably at least the following reflections: 10.6, 17.8, 19.1, 19.4, 25.5, more preferably at least the following reflections: 10.6, 13.9, 17.8, 19.1, 19.4, 23.4, 25.5, most preferably at least the following reflections: 10.6, 13.9, 17.8, 19.1, 19.4, 20.8, 22.0, 22.6, 23.4 and 25.5, each quoted as 2⊖ value ±0.2°. The compound of the formula (I) in the crystalline modification I can also be characterized by the X-Ray powder diffractogram (at 20±5° C. and with Cu—K alpha 1 as radiation) as shown in FIG. 11.

The crystalline modification II of the compound of the formula (I) can be characterized by a X-Ray powder diffractogram (at 20±5° C. and with Cu—K alpha 1 as radiation) which displays at least the following reflections: 11.0, 16.8, 23.6, preferably at least the following reflections: 8.9, 11.0, 16.8, 20.2, 23.6, more preferably at least the following reflections: 7.9, 8.9, 11.0, 16.8, 18.3, 20.2, 23.6, most preferably at least the following reflections: 7.9, 8.9, 11.0, 16.8, 17.3, 18.3, 20.2, 21.9, 23.6 and 26.5, each quoted as 2⊖ value ±0.2°. The compound of the formula (I) in the crystalline modification I can also be characterized by the X-Ray powder diffractogram (at 20±5° C. and with Cu—K alpha 1 as radiation) as shown in FIG. 12.

### PROCESS FOR PREPARING

The invention further relates to a process for the preparation of the compound of the formula (I) in the crystalline modification I, by dissolving the compound of the formula (I) in the amorphous form in an inert solvent and crystallising the compound of the formula (I) in the crystalline modification I with a seed of the compound of the formula (II) in the crystalline modification A.

Inert solvents according to the present invention are acetonitrile, tetrahydrofuran, acetone, ethyl acetate, isopropyl acetate, butyl acetate, butan-2-one, 1,4-dioxane, 2-methylpyridine, 4-methylpentan-2-one, n-heptane, cyclohexane, methylcyclohexane, 2-(propan-2-yloxy)propane or 2-methoxy-2-methylpropane, or alcohols such as butan-1-ol, butan-2-ol, propan-2-ol, propan-1-ol, 2-methylpropan-1-ol, ethanol or methanol, and/or mixtures thereof as well as mixtures of the solvents with water. Preferred as solvent is a mixture of ethanol and water.

The invention further relates to a process for the preparation of the compound of the formula (I) in the crystalline modification I, by dissolving the compound of the formula (I) in the amorphous form in ethanol and adding water and crystallising the compound of the formula (I) in the crystalline modification I with a seed of the compound of the formula (II) in the crystalline modification A.

Compound of the formula (II), 4-({(2S)-2-[4-{3-chloro-2-fluoro-6-[4-(trifluoromethyl)-1H-1,2,3-triazol-1-yl]phenyl}-5-methoxy-2-oxopyridin-1(2H)-yl]propanoyl}amino)-2-fluorobenzamide, has the following formula:

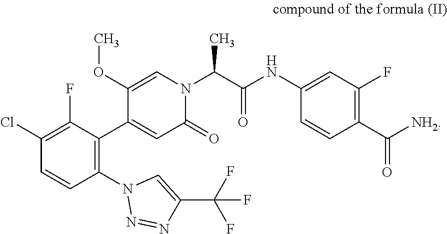 [SMILES: COc1cn([C@@H](C)C(=O)Nc2ccc(C(N)=O)c(F)c2)c(=O)cc1-c1c(-n2cc(C(F)(F)F)nn2)ccc(Cl)c1F]

The invention further relates to a process for the preparation of the compound of the formula (I) in the crystalline modification II, by drying the compound of the formula (III) in an oven under reduced pressure, preferable for one day at 50° C. and 10 mbar. Other combinations of temperature and pressure can also lead to desolvation of acetone, whereby the progress and/or conclusion of the desolvation process can be verified by TGA and XRPD measurements.

Compound of the formula (III), 4-({(2S)-2-[4-{5-chloro-2-[4-(trifluoromethyl)-1H-1,2,3-triazol-1-yl]phenyl}-5-methoxy-2-oxopyridin-1(2H)-yl]butanoyl}-amino)-2-fluorobenzamide acetone, has the following formula:

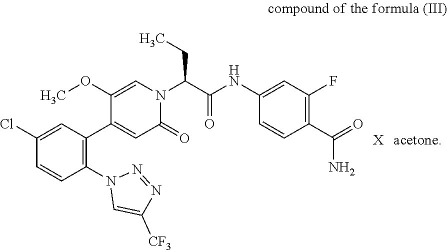

### METHOD FOR TREATMENT

The present invention further relates to the use of the compound of the formula (I) in the crystalline modification I and/or in the crystalline modification II for the treatment and/or prophylaxis of diseases, preferably of thrombotic or thromboembolic disorders and/or thrombotic or thromboembolic complications.

The present invention further relates to the use of the compound of the formula (I) in the crystalline modification I and/or in the crystalline modification II for the treatment and/or prophylaxis of cardiovascular disorders including coronary artery disease, angina pectoris, myocardial infarction or stent thrombosis, as well as disorders in the cerebrovascular arteries and other disorders, leading to transitory ischaemic attacks (TIA), ischemic strokes including cardioembolic as well as non-cardioembolic strokes, and/or disorders of peripheral arteries, leading to peripheral artery disease, including peripheral artery occlusion, acute limb ischemia, amputation, reocclusions and restenoses after interventions such as angioplasty, stent implantation or surgery and bypass, and/or stent thrombosis.

### PHARMACEUTICAL COMPOSITIONS

It is possible for the crystalline modification I and the crystalline modification II of the compound of the formula (I) according to the present invention to have systemic and/or local activity. For this purpose, it can be administered in a suitable manner, such as, for example, via the oral, parenteral, pulmonary, nasal, sublingual, lingual, buccal, rectal, vaginal, dermal, transdermal, conjunctival, otic route or as an implant or stent.

For these administration routes, it is possible for the crystalline modification I and the crystalline modification II of the compound of the formula (I) according to the present invention to be administered in suitable administration forms.

For oral administration, it is possible to formulate the crystalline modification I and the crystalline modification II of the compound of the formula (I) according to the present invention to dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophilisates, capsules (for example hard or soft gelatin capsules), sugar-coated tablets, granules, pellets, powders, emulsions, suspensions, aerosols or solutions. It is possible to incorporate the compound according to the invention in crystalline and/or amorphous and/or dissolved form into said dosage forms.

Parenteral administration can be effected with avoidance of an absorption step (for example intravenous, intraarterial, intracardial, intraspinal or intralumbal) or with inclusion of absorption (for example intramuscular, subcutaneous, intracutaneous, percutaneous or intraperitoneal). Administration forms which are suitable for parenteral administration are, inter alia, preparations for injection and infusion in the form of solutions, suspensions, emulsions, lyophylisates or sterile powders.

Examples which are suitable for other administration routes are pharmaceutical forms for inhalation [inter alia powder inhalers, nebulizers], nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixturae agitandae), lipophilic suspensions, emulsions, ointments, creams, transdermal therapeutic systems (such as, for example, patches), milk, pastes, foams, dusting powders, implants or stents.

The crystalline modification I and the crystalline modification II of the compound of the formula (I) can be incorporated into the stated administration forms. This can be effected in a manner known per se by mixing with pharmaceutically suitable excipients. Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropyl-cellulose, carboxymethylcellulose-sodium, starch, carbomers, polyacrylic acids (such as, for example, Carbopol®); alginates, gelatine),
disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropyl-methylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

The present invention furthermore relates to a pharmaceutical composition which comprise at least the crystalline modification I and/or the crystalline modification II of the compound of the formula (I) according to the present invention, conventionally together with one or more pharmaceutically suitable excipient(s), and to their use according to the present invention.

### DOSAGE OF THE PHARMACEUTICAL COMPOSITIONS OF THE PRESENT INVENTION

Based upon laboratory techniques known to evaluate compounds useful for the treatment of disorders, by pharmacological assays for the determination of treatment of the conditions identified above in mammals, and by comparison of these results with the results of known medicaments that are used to treat these conditions, the effective dosage of the compound of this invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 5 to 250 mg every 24 hours for parenteral administration to achieve effective results and from about 5 to 500 mg every 24 hours for oral administration to achieve effective results.

In spite of this, it may be necessary, if appropriate, to deviate from the amounts specified, specifically depending on body weight, administration route, individual behaviour towards the active ingredient, type of formulation, and time or interval of administration.

The weight data in the tests and examples which follow are, unless stated otherwise, percentages by weight; parts are parts by weight. Solvent ratios, dilution ratios and concentration data of liquid/liquid solutions are based on each case on the volume, unless otherwise stated.

### WORKING EXAMPLES

Abbreviations:

| | |
|---|---|
| br s | broad singlet (in NMR) |
| br d | broad doublet (in NMR) |
| br t | broad triplet (in NMR) |
| d | day(s), doublet (in NMR) |
| DCI | direct chemical ionization (in MS) |
| dd | doublet of doublets (in NMR) |
| DMSO | dimethyl sulfoxide |
| eq. | equivalent(s) |
| ESI | electrospray ionization (in MS) |
| h | hour(s) |
| HPLC | high-pressure, high-performance liquid chromatography |
| LC/MS | liquid chromatography-coupled mass spectroscopy |
| m | multiplet (in NMR) |
| min | minute(s) |
| MS | mass spectroscopy |
| NMR | nuclear magnetic resonance spectroscopy |
| q | quartet or quadruplet (in NMR) |
| RP | reverse phase (in HPLC) |
| RT | room temperature |
| Rₜ | retention time (in HPLC) |
| s | singlet (in NMR) |
| t | triplet (in NMR) |
| T3P | 2,4,6-tripropyl-1,3,5,2,4,6-trioxatriphosphinane 2,4,6-trioxide |

### HPLC, LC-MS and GC Methods

Method 1: Instrument: Waters ACQUITY SQD UPLC system; column: Waters Acquity UPLC HSS T3 C18 1.8 μm, 50 mm×1.0 mm; eluent A: water+0.025% formic acid, eluent B: acetonitrile+0.025% formic acid; gradient: 0.0 min 10% B→1.2 min 95% B→2.0 min 95% B: oven: 50° C.; flow rate: 0.40 ml/min; UV detection: 210-400 nm.

Method 2: Instrument: Thermo Scientific FT-MS; UHPLC: Thermo Scientific UltiMate 3000; column: Waters HSS T3 C18 1.8 μm, 75 mm×2.1 mm; eluent A: water+0.01% formic acid; eluent B: acetonitrile+0.01% formic acid; gradient: 0.0 min 10% B→2.5 min 95% B→3.5 min 95% B: oven: 50° C.; flow rate: 0.90 ml/min; UV detection: 210-400 nm.

Method 3: Agilent 1290 system; column: YMC Triart C18 ExRS 1.9 82 m, 50 mm×2 mm; eluent A: aqueous ammonium acetate (0.77 g/L)/ammoniac buffer solution pH 9; eluent B: acetonitrile; gradient: 0.0 min 5% B→10 min 65% B→10.01 min 5% B→11 min 5% B: oven: 40° C.; flow rate: 1 ml/min; UV detection: 220 nm.

¹H-NMR method: ¹H-NMR spectra were acquired on Bruker spectrometers (at 400 MHz, 500 MHz or 600 MHz as indicated) at room temperature in deuterated solvent (d₆-DMSO). Information about the chemical shift δ is given in ppm, relative to the irradiation frequency. The signal of the deuterated solvent is used as internal standard.

### Example 1: Preparation of (4S)-2⁴-chloro-4-ethyl-7³-fluoro-3⁵-methoxy-3²,5-dioxo-1⁴-(trifluoromethyl)-3²H-6-aza-3(4,1)-pyridina-1(1)-[1,2,3]triazola-2(1,2),7(1)- dibenzenaheptaphane-7⁴-carboxamide, also Named as 4-({(2S)-2-[4-{5-chloro-2-[4-(trifluoromethyl)-1H-1,2,3-triazol-1-yl]phenyl}-5-methoxy-2-oxopyridin-1(2H)-yl]butanoyl}amino)-2-fluorobenzamide, (Compound of the Formula (I))

The compound of the formula (I) can be prepared as described in WO2017/005725 in Example 234 and Example 235. Using the described process the compound of the formula (I) is obtained in the amorphous form.

The ¹H-NMR of the compound of the formula (I) as racemate is shown in WO2017/005725 in Example 234:

¹H-NMR (400 MHZ, DMSO-d₆): δ [ppm]=10.76 (br s, 1H), 9.13 (s, 1H), 7.86-7.80 (m, 2H), 7.79-7.77 (m, 1H), 7.69 (t, 1H), 7.66-7.61 (m, 1H), 7.56-7.49 (m, 2H), 7.37 (dd, 1H), 7.13 (s, 1H), 6.53 (s, 1H), 5.55-5.49 (m, 1H), 3.26 (s, 3H), 2.14-2.02 (m, 2H), 0.79 (t, 3H).

### Example 2: Preparation of 4-({(2S)-2-[4-{3-Chloro-2-fluoro-6-[4-(trifluoromethyl)-1H-1,2,3triazol-1-yl]phenyl}-5-methoxy-2-oxopyridin-1(2H)-yl]propanoyl}amino)-2-fluoro-benzamide (Compound of the Formula (II))

### Example 2.1: 1-(2-Bromo-4-chloro-3-fluorophenyl)-4-(trifluoromethyl)-1H-1,2,3-triazole

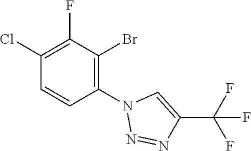 [SMILES: Fc1c(Cl)ccc(-n2cc(C(F)(F)F)nn2)c1Br]

1-(2-Bromo-4-chloro-3-fluorophenyl)-4-(trifluoromethyl)-1H-1,2,3-triazole is synthesized starting with 2-bromo-4-chloro-3-fluoroaniline (WO 2016/168098, page 59-60) by first generating the azido derivative (in the presence of tert-butyl nitrite and trimethylsilyl azide, in analogy to the synthesis of example 2.18A, WO 2017/005725, page 92-93) and second performing a cycloaddition of the azido derivative with trifluoropropyne (in the presence of copper(I) oxide, in analogy to the synthesis of example 2.26A, WO 2017/005725, page 102).

### Example 2.2: 4-{3-Chloro-2-fluoro-6-[4-(trifluoromethyl)-1H-1,2,3-triazol-1-yl]phenyl}-2,5-dimethoxypyridine

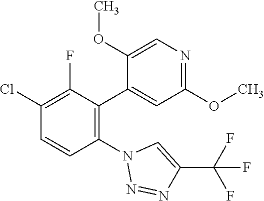 [SMILES: COc1cc(-c2c(-n3cc(C(F)(F)F)nn3)ccc(Cl)c2F)c(OC)cn1]

A mixture of 1-(2-bromo-4-chloro-3-fluorophenyl)-4-(trifluoromethyl)-1H-1,2,3-triazole (982 mg, 2.85 mmol), (2,5-dimethoxypyridin-4-yl)boronic acid (WO 2019/175043, page 23-24) (626 mg, 3.42 mmol, 1.2 eq.) and potassium carbonate (1.18 g, 8.55 mmol, 3.0 eq.) was dissolved in 1,4-dioxane (50 ml) and flushed with argon for 10 min before [1,1-bis(diphenylphosphino)ferrocene]palladium(II) chloride monodichloromethane adduct (233 mg, 0.29 mmol, 0.1 eq.) was added. The reaction mixture was stirred at 100° C. (oil bath already pre-heated to 100° C.) overnight. Additional (2,5-dimethoxypyridin-4-yl)boronic acid (209 mg, 1.14 mmol, 0.4 eq.) and [1,1-bis(diphenylphosphino)ferrocene]palladium(II) chloride monodichloromethane adduct (116 mg, 0.14 mmol, 0.05 eq.) were added. The reaction mixture was stirred at 100° C. for additional 5 h, left at RT for the weekend and filtered through Celite® which was washed with 1,4-dioxane. The combined filtrates were concentrated under reduced pressure. The residue was purified by chromatography (silica gel, eluent: cyclohexane/ethyl acetate gradient). Yield: 432 mg (38% of theory).

LC-MS (method 2): Rₜ=2.13 min; MS (ESIpos): m/z=403 [M+H]⁺

¹H-NMR (400 MHZ, DMSO-d₆): δ [ppm]=9.17/9.16 (2x s, 1H), 8.03/8.01 (2x d, 1H), 7.86 (s, 1H), 7.75/7.75 (2x d, 1H), 6.82 (s, 1H), 3.79 (s, 3H), 3.54 (s, 3H).

### Example 2.3: 4-{3-Chloro-2-fluoro-6-[4-(trifluoromethyl)-1H-1,2,3-triazol-1-yl]phenyl}-5-methoxypyridin-2(1H)-One

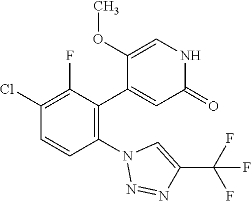 [SMILES: COc1c[nH]c(=O)cc1-c1c(-n2cc(C(F)(F)F)nn2)ccc(Cl)c1F]

Pyridine hydrobromide (429 mg, 2.68 mmol, 2.5 eq.) was added to a solution of 4-{3-chloro-2-fluoro-6-[4-(trifluoromethyl)-1H-1,2,3-triazol-1-yl]phenyl}-2,5-dimethoxypyridine (432 mg, 1.07 mmol) in N,N-dimethylformamide (10 ml). The mixture was stirred at 100° C. overnight and concentrated under reduced pressure. The residue was dissolved in water. After addition of ethyl acetate and phase separation, the aqueous phase was extracted two times with ethyl acetate. The combined organic phases were dried over anhydrous sodium sulfate, filtered and concentrated under reduced pressure. The residue was purified by chromatography (silica gel, eluent: dichloromethane/methanol gradient). Yield: 285 mg (68% of theory).

LC-MS (method 2): Rₜ=1.46 min; MS (ESIpos): m/z=389 [M+H]⁺

¹H-NMR (600 MHZ, DMSO-d₆): δ [ppm]=11.3 (br s, 1H), 9.23 (s, 1H), 8.10-7.99 (m, 1H), 7.77 (m, 1H), 7.15 (s, 1H), 6.41 (s, 1H), 3.45 (s, 3H).

### Example 2.4: 4-({(2S)-2-[4-{3-Chloro-2-fluoro-6-[4-(trifluoromethyl)-1H-1,2,3-triazol-1-yl]phenyl}-5-methoxy-2-oxopyridin-1(2H)-yl]propanoyl}amino)-2-fluorobenzamide (Compound of the Formula (II))

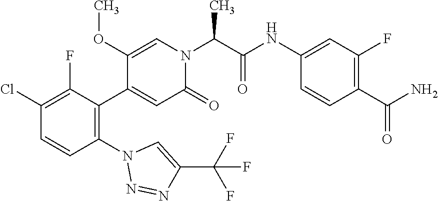 [SMILES: COc1cn([C@@H](C)C(=O)Nc2ccc(C(N)=O)c(F)c2)c(=O)cc1-c1c(-n2cc(C(F)(F)F)nn2)ccc(Cl)c1F]

1,1,3,3-Tetramethylguanidine (420 μl, 3.35 mmol, 3.0 eq.) was added under argon atmosphere at RT to a solution of 4-{3-chloro-2-fluoro-6-[4-(trifluoromethyl)-1H-1,2,3-triazol-1-yl]phenyl}-5-ethoxypyridin-2(1H)-one (438 mg, 1.12 mmol) in 2-propanol/acetone (4:1, 7.5 ml). The mixture was stirred at RT for 15 min, followed by addition of 4-{[(2R)-2-bromopropanoyl]amino}-2-fluorobenzamide (WO 2020/127504, example 1.19A, page 76) (355 mg, 1.23 mmol, 1.1 eq.) and further 2-propanol/acetone (4:1, 7.5 ml). The reaction mixture was stirred at RT overnight and concentrated under reduced pressure. The residue was purified by chromatography (silica gel, eluent: dichloromethane/methanol gradient) and preparative HPLC (reversed phase, eluent: acetonitrile/water gradient). Yield: 539 mg (81% of theory).

LC-MS (method 2): Rₜ=1.65 min; MS (ESIpos): m/z=597 [M+H]⁺

¹H-NMR (500 MHZ, DMSO-d₆): δ [ppm]=10.72/10.63 (2x s, 1H), 9.24/9.13 (2x s, 1H), 8.06-7.99 (m, 1H), 7.79-7.74 (m, 1H), 7.72-7.60 (m, 2H), 7.56-7.48 (m, 2H), 7.38-7.32 (m, 1H), 7.27/7.25 (2x s, 1H), 6.48/6.47 (2x s, 1H), 5.51-5.44 (m, 1H), 3.47/3.45 (2x s, 3H), 1.65/1.64 (2x s, 3H).

### Example 3: Preparation of 4-({(2S)-2-[4-{5-chloro-2-[4-(trifluoromethyl)-1H-1,2,3-triazol-1-yl]phenyl}-5-methoxy-2-oxopyridin-1(2H)-yl]butanoyl}-amino)-2-fluorobenzamide acetone (Compound of the Formula (III)

The compound of the formula (III) can be prepared as described in WO2019/175043 compound of the formula (IIc). Using the described process the compound of the formula (III) is obtained in the crystalline form.

### Example 4: Preparation of the Compound of the Formula (II) in Crystalline Modification A

306 mg of compound of the formula (II) in amorphous form was dissolved in 20 mL of a mixture of 50 vol.-% ethanol and 50 vol.-% water at room temperature. The solution was stirred 24 hours at room temperature, resulting in the precipitation of a white solid. The solvent was evaporated in a rotary evaporator. The obtained solid was dried in a vacuum oven at 40° C. for 16 hours. 273 mg of compound of the formula (II) in the crystalline modification A was obtained. The ¹H-NMR spectrum (in DMSO-d₆) is shown in FIG. 19.

### Example 5: Attempt to Prepare the Compound of the Formula (I) in a Crystalline Modification

Approximately 10 mg compound of the formula (I) in amorphous form was dissolved in 1 mL of hot ethanol. After cooling to room temperature, the solution was stirred in an open vial until the solvent was completely evaporated. The obtained solid was amorphous.

### Example 6: Attempt to Prepare the Compound of the Formula (I) in a Crystalline Mmodification

100 mg compound of the formula (I) in amorphous form was suspended in 2.5 mL of a mixture of 50 vol.-% ethanol and 50 vol.-% water at room temperature. The suspension was stirred 4 weeks, then filtered and dried. The obtained solid was amorphous.

### Example 7: Preparation of the Compound of the Formula (I) in Crystalline Modification I

30 mg compound of the formula (I) in amorphous form was dissolved in 2 mL of ethanol at room temperature. 660 μL of water was added to the solution dropwise until a cloudy solution was observed. The solution was then seeded with 1 mg of crystalline modification A of compound of the formula (II). Shortly after seeding, the precipitation of further small particles was observed, but the particles rapidly disappeared upon stirring, resulting in a seemingly clear solution. After stirring at room temperature for 48 hours, a suspension was obtained. The solid was filtered under vacuum and dried overnight under ambient conditions. The XRPD pattern of the obtained solid corresponds to the crystalline modification I of compound of the formula (I). The ¹H-NMR analysis of the resulting solid indicates that the solid contained approximately 5 wt-% of compound of the formula (II). Peaks of the compound of the formula (I) are at δ [ppm]=6.53 (s, 1H), 3.26 (s, 3H) and 0.79 (t, 3H) and peaks of the compound of the formula (II) are at δ [ppm]=6.48/6.47 (2x s, 1H), 3.47/3.45 (2x s, 3H) and 1.65/1.64 (2x s, 3H). These peaks were used for integration in order to determine the 5 wt-% of the compound of the formula (II). The ¹H-NMR spectrum is shown in FIG. 1.

### Example 8: Preparation of the Compound of the Formula (I) in Crystalline Modification I as Pure Compound of the Formula (I)

300 mg compound of the formula (I) in amorphous form was dissolved in 3.8 mL of ethanol at room temperature. 3.5 mL of water was added to the solution dropwise until a cloudy solution was observed. 2 drops of ethanol were added to obtain a clear solution. The clear solution was seeded with 1.5 mg of the solid obtained in example 7, then stirred at room temperature for 2 days. The resulting suspension was filtered and dried over night at ambient conditions. 146 mg of the crystalline modification I of compound of the formula (I) was obtained. The ¹H-NMR analysis of the resulting solid indicates that the amount of compound of the formula (II) was below the detection limit. The ¹H-NMR spectrum is shown in FIG. 2.

### Example 9: Preparation of the Compound of the Formula (I) in Crystalline Modification I as Pure Compound of the Formula (I)

20.0 g compound of the formula (I) in amorphous form was dissolved in a mixture of 40.0 g of propan-2-ol and 10.0 g of acetone, at room temperature. The mixture was heated up to 60° C. and to the resulting solution 126.0 g of water was added during 60 minutes. The resulting mixture was seeded with 100.0 mg of crystalline modification I of compound of the formula (I) and stirred at 60° C. for 3 hours. An additional 4.8 g of compound of the formula (I) in amorphous form was then added and the mixture was stirred at 60° C. overnight. The resulting suspension was cooled down to 20° C. in 60 minutes and stirred at 20° C. for 90 minutes. So-obtained suspension was filtered under vacuum, washed twice with 42.5 g of propan-2-ol : acetone : water mixture in the mass ratio 4:1:12 and dried in vacuum, at 40° C. Yield: 22.4 g (90.3% of theoretical yield) of pale-white solid in the crystalline modification I.

### Example 10: Preparation of the Compound of the Formula (I) in Crystalline Modification II

40 mg of the compound of the formula (III) was dried at 50° C. under reduced pressure to obtain solid in the crystalline modification II.

### Example 11: Physical Characterization of Amorphous Form, Crystalline Modification I and Crystalline Modification II of the Compound of the Formula (I)

### Example 11.1: Thermogravimetric Analysis (TGA)

Thermogravimetric analysis (TGA) was performed with either a Perkin Elmer Pyris 6 or a Mettler Toledo TGA/DSC1. The instrument was purged with nitrogen gas at a flow rate of 20-50 ml.min⁻¹. Approximately 5-15 mg of each sample was placed into either an aluminum or an aluminum oxide crucible. The heating rate was 10° C.min⁻¹ for all measurements, with a temperature range of 25-300° C. for Modification I and II, and a temperature range of 25-280° C. for the amorphous form. No sample preparation was conducted. TGA thermograms are shown in FIGS. 3 and 4 and 15.

### Example 11.2: Differential Scanning Calorimetry (DSC)

### FIG. 16: DSC Curve of Compound of the Formula (I), Amorphous Form

Differential scanning calorimetry (DSC) was performed with a Mettler Toledo DSC822e. The calorimeter was purged with nitrogen gas at a flow rate of 50 ml.min⁻¹. Approximately 3-10 mg of sample was placed into an aluminum crucible without sample preparation. The temperature range was −10-280° C. at a heating rate of 20° C.min⁻¹. The DSC thermogram is shown in FIG. 16.

### FIG. 5: DSC Curve of Compound of the Formula (I), Crystalline Modification I

Differential scanning calorimetry (DSC) was performed with a Mettler Toledo DSC3. The calorimeter was purged with nitrogen gas at a flow rate of 50 ml.min⁻¹. Approximately 3-10 mg of sample was placed into an aluminum crucible without sample preparation. The temperature range was −10-300° C. at a heating rate of 20° C.min⁻¹. The DSC thermogram is shown in FIG. 5.

### FIG. 6: DSC Curve of Compound of the Formula (I), Crystalline Modification II

Differential scanning calorimetry (DSC) was performed with a Netzsch Phoenix DSC 204 F1. The calorimeter was purged with nitrogen gas at a flow rate of 20 ml.min⁻¹. Approximately 3-10 mg of sample was placed into an aluminum crucible without sample preparation. The temperature range was 25-300° C. at a heating rate of 10° C.min⁻¹. The DSC thermogram is shown in FIG. 6.

TABLE 1
Differential scanning calorimetry

| | crystalline | crystalline |
|---|---|---|
| | modification I | modification II |
| Melting point [° C.] | 196° C. | — |

### Example 11.3: Infrared Spectroscopy

IR measurements were performed with a Thermo Scientific Nicolet iS10 spectrometer and a Bruker alpha spectrometer in the attenuated total reflectance (ATR) geometry. No sample preparation was performed. and each individual measurement consisted of 3²or 64 scans. IR spectra are shown in FIGS. 7 and 8.

TABLE 2
Infrared spectroscopy of the compound of the formula (I),
crystalline modification I and crystalline modification II

| Band maxima (cm⁻¹) | | Band maxima (cm⁻¹) | |
|---|---|---|---|
| Crystalline | Crystalline | Crystalline | Crystalline |
| modification I | modification I | modification II | modification II |
| 402 | 996 | 619 | 2967 |
| 427 | 1039 | 675 | 3107 |
| 440 | 1069 | 710 | 3166 |
| 459 | 1096 | 727 | 3293 |
| 502 | 1129 | 746 | 3509 |
| 521 | 1141 | 775 | |
| 535 | 1158 | 792 | |
| 568 | 1205 | 825 | |
| 578 | 1234 | 870 | |
| 608 | 1259 | 911 | |
| 618 | 1328 | 979 | |
| 653 | 1383 | 991 | |
| 677 | 1429 | 1032 | |
| 685 | 1465 | 1076 | |
| 704 | 1503 | 1103 | |
| 726 | 1525 | 1134 | |
| 749 | 1568 | 1223 | |
| 791 | 1579 | 1237 | |
| 801 | 1595 | 1259 | |
| 823 | 1613 | 1292 | |
| 835 | 1641 | 1335 | |
| 843 | 1692 | 1373 | |
| 852 | 1705 | 1417 | |
| 867 | 2933 | 1464 | |
| 878 | 2982 | 1525 | |
| 895 | 3060 | 1571 | |
| 915 | 3174 | 1664 | |
| 969 | 3332 | 2844 | |
| 977 | 3401 | 2938 | |

### Example 11.4: Raman Spectroscopy of the Compound of the Formula (I)

Raman measurements were performed with a Bruker MultiRAM spectrometer. No sample preparation was performed. and each individual measurement consisted of 64 or 128 scans using a laser power of 300 or 600 mW. Raman spectra are shown in FIGS. 9 and 10.

TABLE 3
Raman spectroscopy of the compound of the formula (I), crystalline
modification I and crystalline modification II

| Band maxima (cm⁻¹) | | Band maxima (cm⁻¹) | |
|---|---|---|---|
| Crystalline | Crystalline | Crystalline | Crystalline |
| modification I | modification I | modification II | modification II |
| 226 | 1125 | 229 | 1663 |
| 238 | 1138 | 337 | 1710 |
| 252 | 1210 | 391 | 2844 |
| 271 | 1239 | 425 | 2874 |
| 316 | 1259 | 448 | 2942 |
| 370 | 1273 | 493 | 3075 |
| 381 | 1293 | 622 | |
| 396 | 1329 | 653 | |
| 420 | 1340 | 682 | |
| 460 | 1379 | 709 | |
| 515 | 1395 | 745 | |
| 537 | 1431 | 817 | |
| 558 | 1441 | 880 | |
| 566 | 1473 | 981 | |
| 608 | 1504 | 1037 | |
| 626 | 1528 | 1100 | |
| 640 | 1572 | 1139 | |
| 655 | 1601 | 1183 | |
| 687 | 1625 | 1216 | |
| 698 | 1667 | 1247 | |
| 718 | 1694 | 1274 | |
| 754 | 1706 | 1291 | |
| 792 | 2843 | 1336 | |
| 806 | 2886 | 1374 | |
| 853 | 2916 | 1417 | |
| 866 | 2925 | 1445 | |
| 880 | 2944 | 1486 | |
| 970 | 2960 | 1501 | |
| 991 | 2999 | 1528 | |
| 1034 | 3031 | 1571 | |
| 1070 | 3059 | 1586 | |
| 1079 | 3093 | 1604 | |
| 1100 | | 1623 | |

### Example 11.5: X-Ray Powder Diffraction (XRPD) for Compound of the Formula (I)

X-ray powder diffraction (XRPD) data were recorded on a STOE STADI P or a D8 Bruker Advance diffractometer using monochromatized Cu—K alpha 1 radiation, a position sensitive detector, at generator settings of 40 kV and 40 mA. The samples were collected in transition mode, being either prepared into a standard glass capillary or as a thin layer between two foils. The scanning rage was between 2° and 40° 2 theta with a 0.5° step at 15 seconds/step for the STOE STADI P and a 0.009194171° step at 1.28 seconds/step for the D8 Bruker Advance. X-ray powder diffractograms are shown in FIGS. 11, 12 and 17.

TABLE 4
X-ray powder diffraction (XRPD) of the compound of the formula
(I), crystalline modification I and crystalline modification II

| Diffraction angle (2θ, °) | | Diffraction angle (2θ, °) | |
|---|---|---|---|
| Crystalline | Crystalline | Crystalline | Crystalline |
| modification I | modification I | modification II | modification II |
| 5.8 | 27.5 | 6.0 | 22.6 |
| 8.8 | 28.4 | 7.9 | 22.8 |
| 10.6 | 28.8 | 8.1 | 23.7 |
| 11.6 | 29.5 | 8.9 | 25.2 |
| 13.3 | 29.7 | 9.8 | 25.5 |
| 13.9 | 30.4 | 10.4 | 25.9 |
| 16.0 | 30.7 | 11.0 | 26.5 |
| 17.0 | 31.4 | 11.5 | 27.2 |
| 17.4 | 31.6 | 11.8 | 28.1 |
| 17.8 | 32.5 | 12.2 | 29.0 |
| 18.3 | 33.4 | 13.1 | 29.1 |
| 19.1 | 33.7 | 13.3 | 29.9 |
| 19.4 | 34.3 | 13.8 | 30.3 |
| 20.0 | 34.4 | 14.4 | 30.5 |
| 20.2 | 35.0 | 15.5 | 30.8 |
| 20.8 | 35.5 | 15.8 | 31.7 |
| 21.2 | 35.7 | 16.2 | 32.3 |
| 21.5 | 36.0 | 16.7 | 32.6 |
| 22.0 | 36.6 | 17.3 | 34.4 |
| 22.6 | 37.2 | 17.8 | 35.1 |
| 23.4 | 37.4 | 18.3 | 35.4 |
| 24.0 | 38.1 | 19.0 | 35.9 |
| 24.3 | 38.5 | 19.4 | 36.1 |
| 24.9 | 39.1 | 20.2 | 36.8 |
| 25.5 | 39.6 | 20.6 | 37.3 |
| 26.0 | | 21.4 | 38.1 |
| 26.5 | | 21.9 | |

### Example 11.6: Dynamic Vapour Sorption of the Compound of the Formula (I), Amorphous Form, Crystalline Modification I and Crystalline Modification II

Water sorption isotherms of crystalline modification I and crystalline modification II were determined using a DVS Resolution gravimetric sorption analyzer (London, UK). The water sorption isotherm of the amorphous form was determined using a DVS Intrinsic instrument (Surface Measurement Systems SMS). The sample was dried for 1000 minutes (1340 minutes for the amorphous form) at 0% relative humidity (rH). Afterwards the dry weight was recorded. The humidity was increased in steps of 10% to 90% rH (95% rH for the amorphous form) and then decreased again to 0% rH. The equilibrium criterion for each relative humidity set point was 0.002% per minute relative mass change as a function of time. Dynamic vapour sorption isotherms are shown in FIGS. 13, 14 and 20.

TABLE 5
Dynamic vapour sorption of the compound of the formula (I), amorphous
form, crystalline modification I and crystalline modification II

| | Crystalline modification I | | Crystalline modification II | | Amorphous form | |
|---|---|---|---|---|---|---|
| % rH | Sorption | Desorption | Sorption | Desorption | Sorption | Desorption |
| 0 | 0.0 | 0.0 | 0.02 | 0.01 | 0 | 0.04 |
| 10 | 0.03 | 0.01 | 0.38 | 0.53 | 0.52 | 1.11 |
| 20 | 0.03 | 0.02 | 0.62 | 0.87 | 0.99 | 1.79 |
| 30 | 0.03 | 0.02 | 0.84 | 1.15 | 1.40 | 2.24 |
| 40 | 0.03 | 0.02 | 1.06 | 1.42 | 1.76 | 2.60 |
| 50 | 0.03 | 0.02 | 1.30 | 1.68 | 2.10 | 2.94 |
| 60 | 0.03 | 0.03 | 1.56 | 1.93 | 2.44 | 3.18 |
| 70 | 0.03 | 0.03 | 1.83 | 2.15 | 2.81 | 3.42 |
| 80 | 0.04 | 0.03 | 2.13 | 2.34 | 3.21 | 3.63 |
| 90 | 0.04 | 0.04 | 2.47 | 2.47 | 3.67 | 3.84 |
| 95 | | | | | 3.94 | 3.94 |

### Example 12: X-Ray Powder Diffraction (XRPD) for Compound of the Formula (II), Crystalline Modification A

X-ray powder diffraction (XRPD) data were recorded on a PANalytical X′Pert PRO diffractometer using Cu—K alpha radiation, a position sensitive detector, at generator settings of 40 kV and 40 mA. The samples were collected in transition mode, being prepared as a thin layer between two foils. The scanning rage was between 2° and 40° 2 theta with a 0.013° step at 25 seconds/step. X-ray powder diffractogram is shown in FIG. 18.

TABLE 6
X-ray powder diffraction (XRPD) of the compound
of the formula (II), crystalline modification A
Diffraction angle (2θ, °)
Crystalline modification A

2.75
8.71
12.30
12.95
13.88
15.53
16.01
16.87
17.49
17.94
19.05
19.29
19.84
20.18
21.00
21.43
22.48
23.12
24.33
24.74
26.20
27.06
27.80
29.70
31.38
33.13
33.62

### Example 13: Assessment of Physiological Efficacy of Compound of the Formula (II) (Example 2.4)

The suitability of the compounds according to the invention for treating thromboembolic disorders can be demonstrated in the following assay systems:

### a) Test Descriptions (In Vitro)

### a. 1) Measurement of FXIa Inhibition

The factor XIa inhibition of the substances according to the invention is determined using a biochemical test system which utilizes the reaction of a peptidic factor XIa substrate to determine the enzymatic activity of human factor XIa. Here, factor XIa cleaves from the peptidic factor XIa substrate the C-terminal aminomethylcoumarin (AMC), the fluorescence of which is measured. The determinations are carried out in microtitre plates.

Test substances are dissolved in dimethyl sulfoxide and serially diluted in dimethyl sulfoxide (3000 μM to 0.0078 μM; resulting final concentrations in the test: 50 M to 0.00013 μM). In each case 1 μl of the diluted substance solutions is placed into the wells of white microtitre plates from Greiner (384 wells). 20 μl of assay buffer (50 mM of Tris/HCl pH 7.4; 100 mM of sodium chloride; 5 mM of calcium chloride; 0.1% of bovine serum albumin) and 20 μl of factor XIa from Kordia (0.45 nM in assay buffer) are then added successively. After 15 min of incubation, the enzyme reaction is started by addition of 20 μl of the factor XIa substrate Boc-Glu(OBzl)-Ala-Arg-AMC dissolved in assay buffer (10 μM in assay buffer) from Bachem, the mixture is incubated at room temperature (22° C.) for 30 min and fluorescence is then measured (excitation: 360 nm, emission: 460 nm). The measured emissions of the test batches with test substance are compared to those of control batches without test substance (only dimethyl sulfoxide instead of test substance in dimethyl sulfoxide), and IC₅₀ values are calculated from the concentration/activity relationships. Activity data from this test are listed in Table A below (some as mean values from multiple independent individual determinations):

TABLE A

| Example No. | IC₅₀ [nM] |
|---|---|
| 2.4 | 1.2 |

### a.2) Determination of the Selectivity

To demonstrate the selectivity of the substances with respect to FXIa inhibition, the test substances are examined for their potential to inhibit other human serine proteases, such as factor Xa, trypsin and plasmin. To determine the enzymatic activity of factor Xa (1.3 nmol/l from Kordia), trypsin (83 mU/ml from Sigma) and plasmin (0.1 μg/ml from Kordia), these enzymes are dissolved (50 mmol/l of Tris buffer [C,C,C-tris(hydroxymethyl)aminomethane], 100 mmol/l of NaCl, 0.1% BSA [bovine serum albumin], 5 mmol/l of calcium chloride, pH 7.4) and incubated for 15 min with test substance in various concentrations in dimethyl sulfoxide and also with dimethyl sulfoxide without test substance. The enzymatic reaction is then started by addition of the appropriate substrates (5 μmol/l of Boc-Ile-Glu-Gly-Arg-AMC from Bachem for factor Xa and trypsin, 50 μmol/l of MeOSuc-Ala-Phe-Lys-AMC from Bachem for plasmin). After an incubation time of 30 min at 22° C., fluorescence is measured (excitation: 360 nm, emission: 460 nm). The measured emissions of the test mixtures with test substance are compared to the control mixtures without test substance (only dimethyl sulfoxide instead of test substance in dimethyl sulfoxide) and IC₅₀ values are calculated from the concentration/activity relationships.

### a.3) Thrombin Generation Assay (Thrombogram)

The effect of the test substances in the thrombin generation assay according to Hemker is determined in vitro in human plasma (Octaplas® from Octapharma).

In the thrombin generation assay according to Hemker, the activity of thrombin plasma is determined by measuring the fluorescent cleavage products of the substrate I-1140 (Z-Gly-Gly-Arg-AMC, Bachem). The reactions are carried out in the presence of varying concentrations of test substance or the corresponding solvent. To start the reaction, reagents from Thrombinoscope (30 pM to 0.1 pM recombinant tissue factor, 24 μM phospholipids in HEPES) are used. In addition, a thrombin calibrator from Thrombinoscope is used, of which the amidolytic activity is required for calculating the thrombin activity in a sample containing an unknown amount of thrombin. The test is carried out according to the manufacturer's instructions (Thrombinoscope BV): 4 μl of test substance or of the solvent, 76 μl of plasma and 20 μl of PPP reagent or thrombin calibrator are incubated at 37° C. for 5 min. After addition of 20 μl of 2.5 mM thrombin substrate in 20 mM Hepes, 60 mg/ml of BSA, 102 mM of calcium chloride, the thrombin generation is measured every 20 s over a period of 120 min. Measurement is carried out using a fluorometer (Fluoroskan Ascent) from Thermo Electron fitted with a 390/460 nm filter pair and a dispenser.

Using the Thrombinoscope software, the thrombogram is calculated and represented graphically. The following parameters are calculated: lag time, time to peak, peak, ETP (endogenous thrombin potential) and start tail.

### a.4) Determination of Anticoagulatory Activity

The anticoagulatory activity of the test substances is determined in vitro in human plasma and rat plasma. Fresh whole blood is drawn directly into a mixing ratio of sodium citrate/blood of 1:9 using a 0.11 molar sodium citrate solution as receiver. Immediately after the blood has been drawn, it is mixed thoroughly and centrifuged at about 4000 g for 15 minutes. The supernatant is collected as (platelet-poor) plasma.

The prothrombin time (PT, synonyms: thromboplastin time, quick test) is determined in the presence of varying concentrations of test substance or the corresponding solvent using a commercial test kit (Neoplastin® from Boehringer Mannheim or Hemoliance® RecombiPlastin from Instrumentation Laboratory). The test compounds are incubated with plasma at 37° C. for 3 minutes. Coagulation is then started by addition of thromboplastin, and the timepoint, at which clotting of the sample occurs is determined. The concentration of test substance which effects a doubling of the prothrombin time is determined.

The activated partial thromboplastin time (APTT) is determined in the presence of varying concentrations of test substance or the corresponding solvent using a commercial test kit (PTT reagent from Roche). The test compounds are incubated with the plasma and the PTT reagent (cephalin, kaolin) at 37° C. for 3 minutes. Coagulation is then started by addition of 25 mM calcium chloride, and the time when coagulation occurs is determined. The concentration of test substance which leads to an extension by 50% or a doubling of the APTT is determined.

### a.5) Determination of the Plasma Kallikrein Activity

To determine the plasma kallikrein inhibition of the substances according to the invention, a biochemical test system is used which utilizes the reaction of a peptidic plasma kallikrein substrate to determine the enzymatic activity of human plasma kallikrein. Here, plasma kallikrein cleaves from the peptidic plasma kallikrein substrate the C-terminal aminomethylcoumarin (AMC), the fluorescence of which is measured. The determinations are carried out in microtitre plates.

Test substances are dissolved in dimethyl sulfoxide and serially diluted in dimethyl sulfoxide (3000 μM to 0.0078 M; resulting final concentrations in the test: 50 μM to 0.00013 μM). In each case 1 μl of the diluted substance solutions is placed into the wells of white microtitre plates from Greiner (384 wells). 20 μl of assay buffer (50 mM Tris/HCl pH 7.4; 100 mM sodium chloride solution; 5 mM of calcium chloride solution; 0.1% of bovine serum albumin) and 20 μl of plasma kallikrein from Kordia (0.6 nM in assay buffer) are then added successively. After 15 min of incubation, the enzyme reaction is started by addition of 20 μl of the substrate H-Pro-Phe-Arg-AMC dissolved in assay buffer (10 μM in assay buffer) from Bachem, the mixture is incubated at room temperature (22° C.) for 30 min and fluorescence is then measured (excitation: 360 nm, emission: 460 nm). The measured emissions of the test batches with test substance are compared to those of control batches without test substance (only dimethyl sulfoxide instead of test substance in dimethyl sulfoxide), and IC₅₀ values are calculated from the concentration/activity relationships. Activity data from this test are listed in Table B below (some as mean values from multiple independent individual determinations):

TABLE B

| Example No. | IC₅₀ [nM] |
|---|---|
| 2.4 | 3.4 |

### EXPLANATION OF THE FIGURES

FIG. 1: ¹H NMR of the solid obtained in example 7

FIG. 2: ¹H NMR of the of the solid obtained in example 8

FIG. 3: TGA Curve of compound of the formula (I), crystalline modification I

FIG. 4: TGA Curve of compound of the formula (I), crystalline modification II

FIG. 5: DSC Curve of compound of the formula (I), crystalline modification I

FIG. 6: DSC Curve of compound of the formula (I), crystalline modification II

FIG. 7: IR spectrum of compound of the formula (I), crystalline modification I

FIG. 8: IR spectrum of compound of the formula (I), crystalline modification II

FIG. 9: Raman spectrum of compound of the formula (I), crystalline modification I

FIG. 10: Raman spectrum of compound of the formula (I), crystalline modification II

FIG. 11: X-ray powder diffraction (XRPD) of compound of the formula (I), crystalline modification I

FIG. 12: X-ray powder diffraction (XRPD) of compound of the formula (I), crystalline modification II

FIG. 13: Dynamic vapour sorption of compound of the formula (I), crystalline modification I

FIG. 14: Dynamic vapour sorption of compound of the formula (I), crystalline modification II

FIG. 15: TGA Curve of compound of the formula (I), amorphous form

FIG. 16: DSC Curve of compound of the formula (I), amorphous form

FIG. 17: X-ray powder diffraction (XRPD) of compound of the formula (I), amorphous form

FIG. 18: X-ray powder diffraction (XRPD) of compound of the formula (II), crystalline modification A

FIG. 19: ¹H NMR of the compound of the formula (II), crystalline modification A

FIG. 20: Dynamic vapour sorption of compound of the formula (I), amorphous form

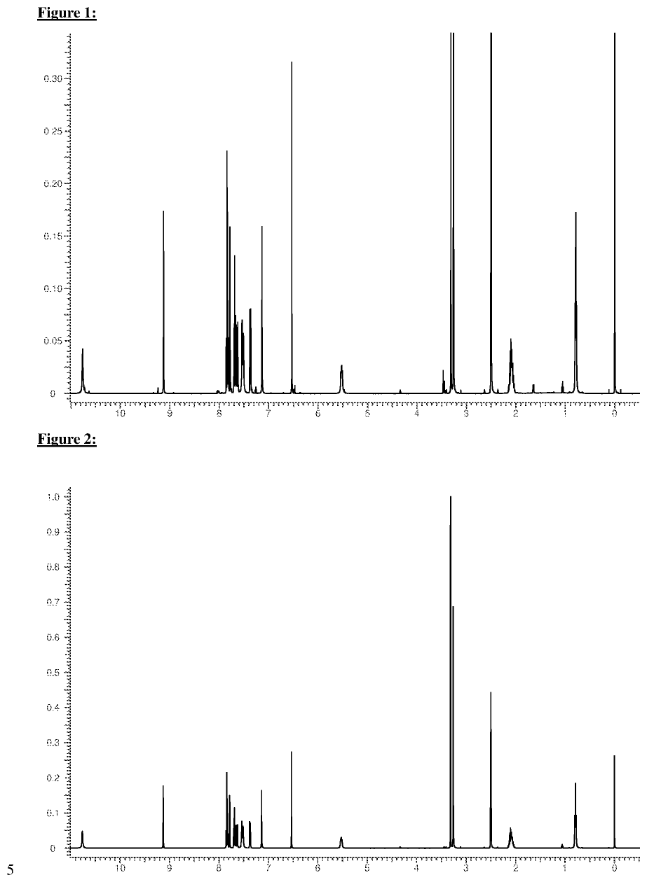

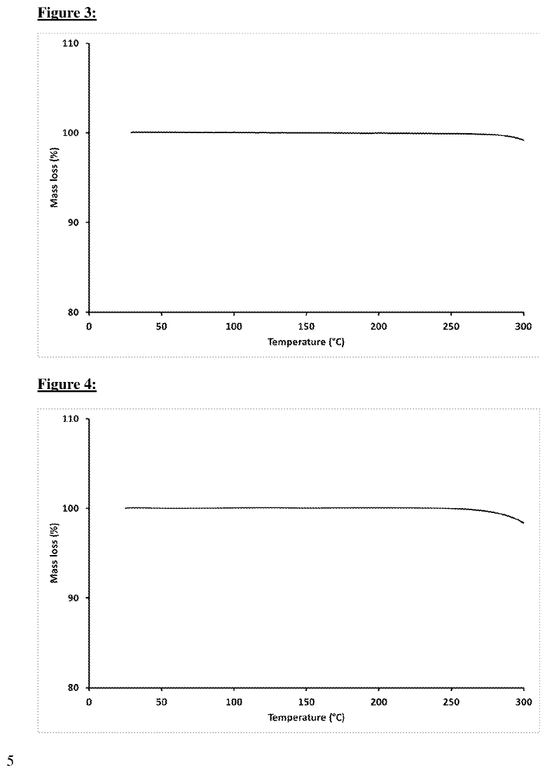

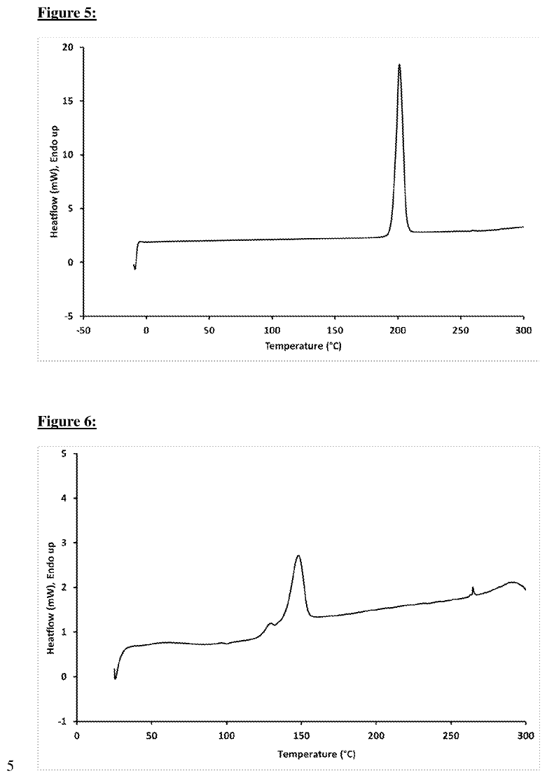

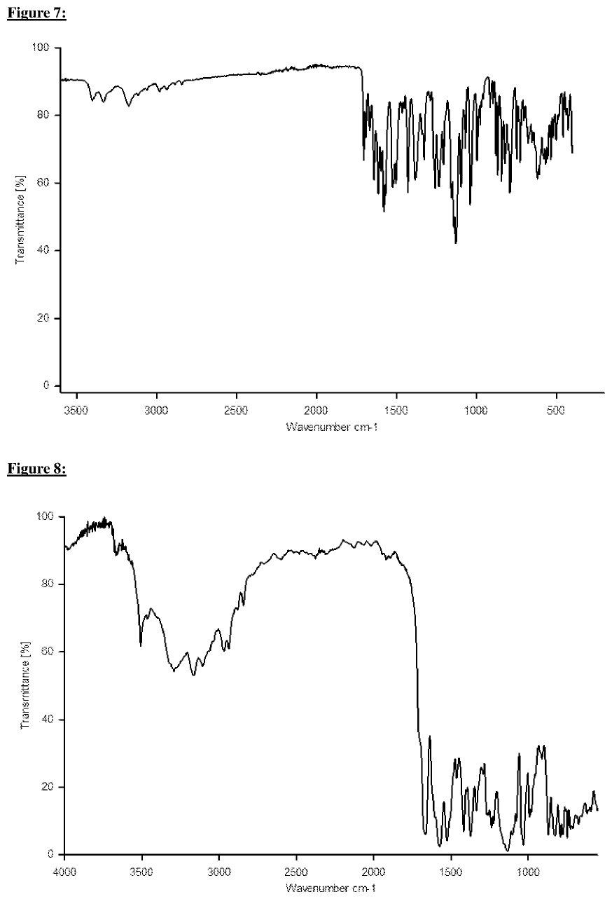

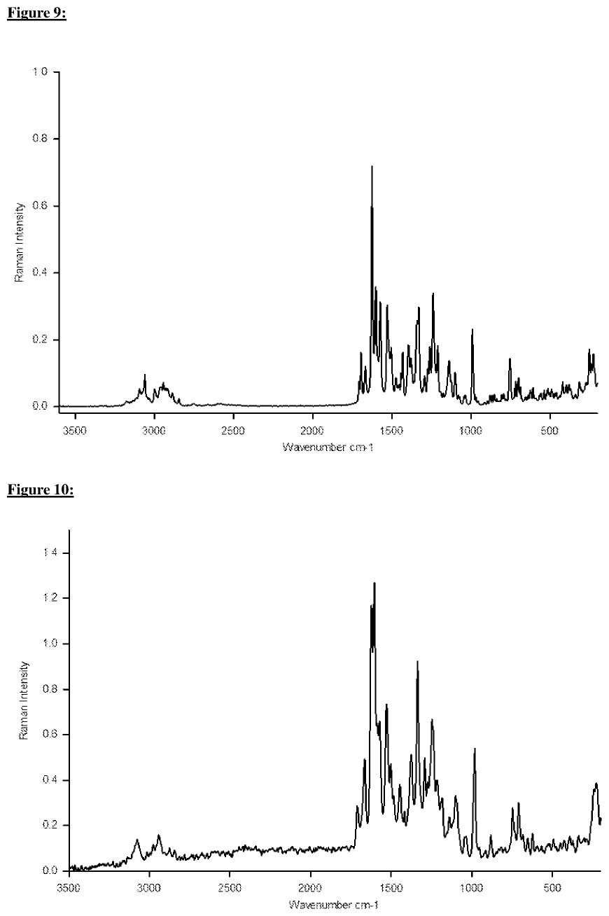

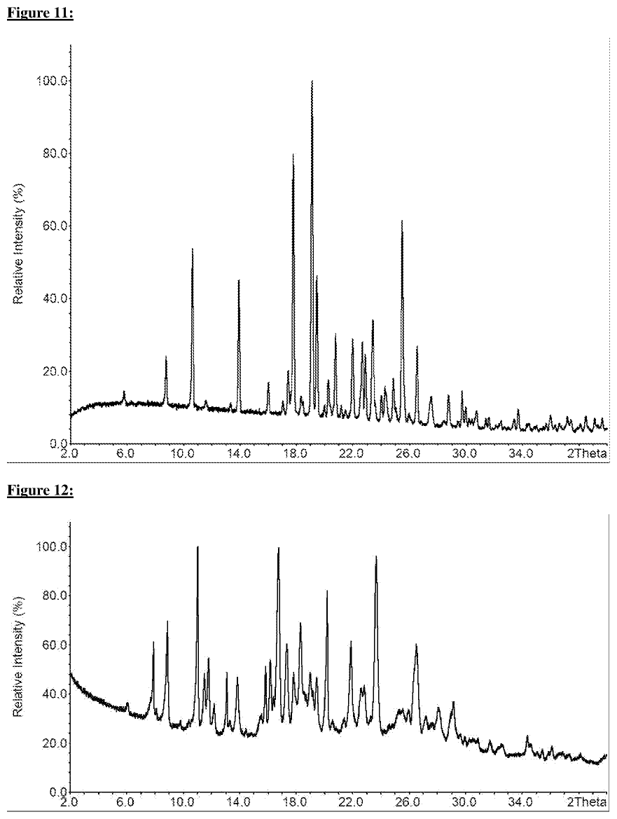

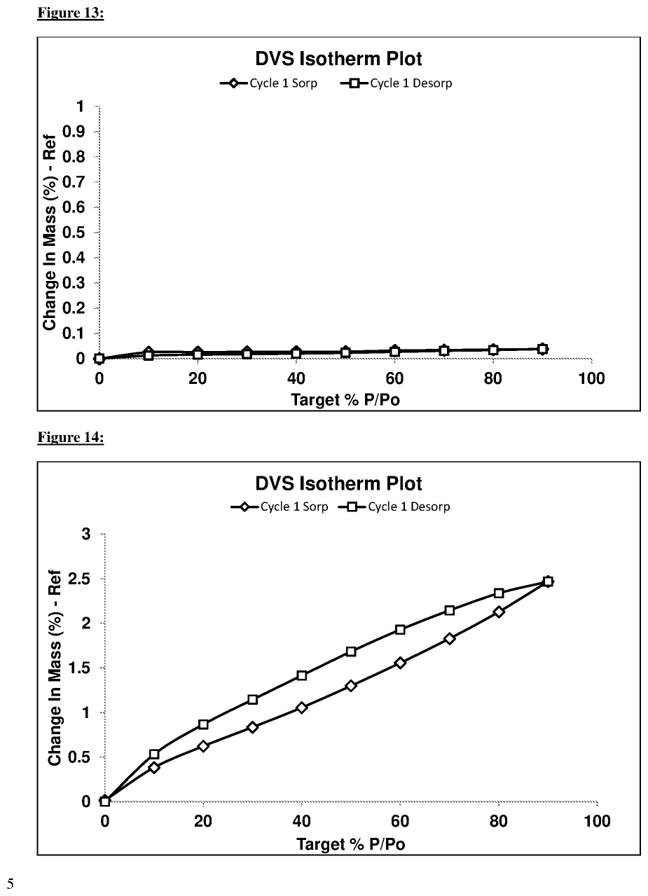

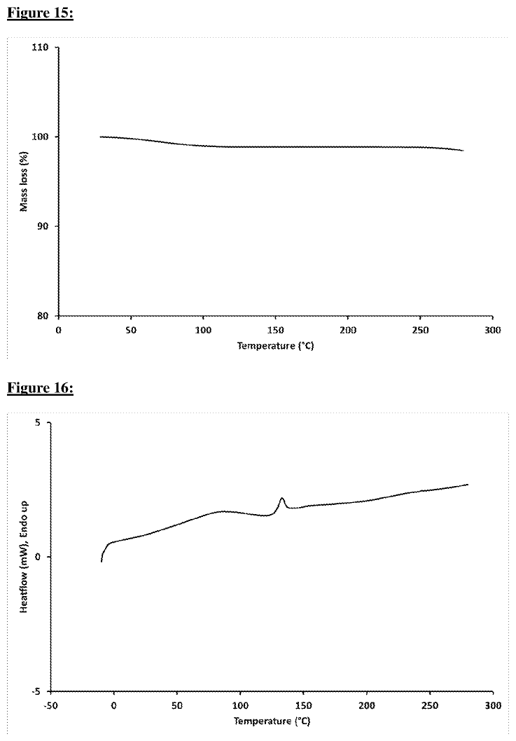

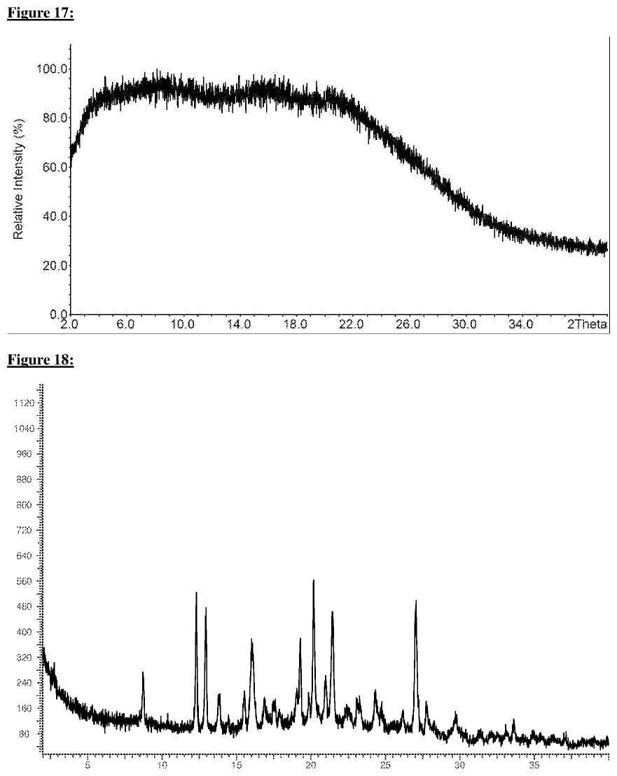

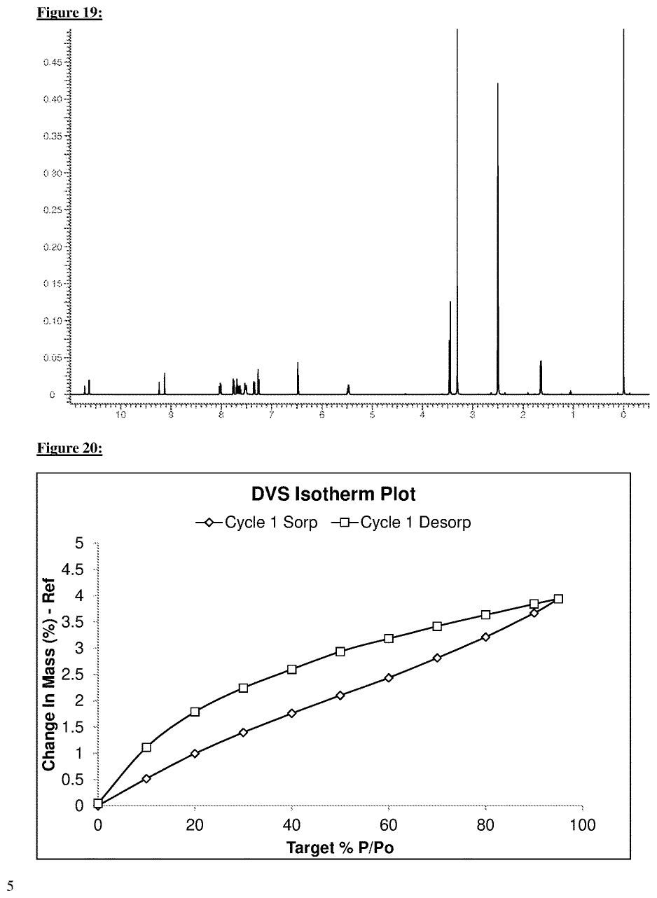

1. A crystalline form of (4S)-2⁴-chloro-4-ethyl-7³-fluoro-3⁵-methoxy-3²,5-dioxo-1⁴-trifluoromethyl)-3²H-6-aza-3(4,1)-pyridina-1(1)-[1,2,3]triazola-2(1,2),7(1)-dibenzenaheptaphane-7⁴-carboxamide having the following formula (I) 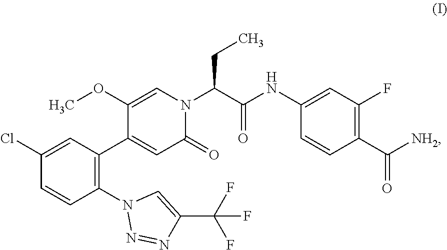 [SMILES: CC[C@@H](C(=O)Nc1ccc(C(N)=O)c(F)c1)n1cc(OC)c(-c2cc(Cl)ccc2-n2cc(C(F)(F)F)nn2)cc1=O]
which is the crystalline modification I or crystalline modification II.

2. The crystalline modification I of the compound of the formula (I) of claim 1 characterized by a X-ray powder diffractogram measured at 20±5° C. and with Cu—K alpha 1 as radiation displaying at least the following reflections, quoted as 2⊖ value ±0.2°: 17.8, 19.1, 25.5.

3. The crystalline modification II of the compound of the formula (I) of claim 1 characterized by a X-ray powder diffractogram measured at 20±5° C. and with Cu—K alpha 1 as radiation displaying at least the following reflections, quoted as 2⊖ value ±0.2°: 11.0, 16.8, 23.6.

4. The crystalline modification I of the compound of the formula (I) of claim 1 characterized by Raman spectroscopy displaying at least the following values of the band maxima (cm⁻¹): 1625, 1239, 991.

5. The crystalline modification II of the compound of the formula (I) of claim 1 characterized by Raman spectroscopy displaying at least the following values of the band maxima (cm⁻¹): 1623, 1604, 1336.

6. The crystalline modification I of the compound of the formula (I) of claim 1 for use in the treatment and/or prophylaxis of thrombotic or thromboembolic disorders and/or thrombotic or thromboembolic complications.

7. The crystalline modification II of the compound of the formula (I) of claim 1 for use in the treatment and/or prophylaxis of thrombotic or thromboembolic disorders and/or thrombotic or thromboembolic complications.

8. Process for preparing the crystalline modification I of the compound of the formula (I) of claim 1, characterized in that the compound of the formula (I) in the amorphous form is dissolved in an inert solvent and the compound of the formula (I) in the crystalline modification I is crystallized with a seed of 4-({(2S)-2-[4-{3-chloro-2-fluoro-6-[4-(trifluoromethyl)-1H-1,2,3-triazol-1-yl]phenyl}-5-methoxy-2-oxopyridin-1(2H)-yl]-propanoyl}amino)-2-fluorobenzamide in the crystalline modification A having the following formula (II)

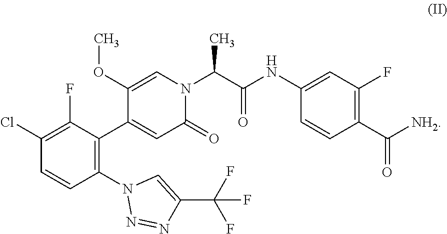 [SMILES: COc1cn([C@@H](C)C(=O)Nc2ccc(C(N)=O)c(F)c2)c(=O)cc1-c1c(-n2cc(C(F)(F)F)nn2)ccc(Cl)c1F]

9. The method according to claim 8, characterized in that the inert solvent is selected from the group consisting of acetonitrile, tetrahydrofuran, acetone, ethyl acetate, isopropyl acetate, butyl acetate, butan-2-one, 1,4-dioxane, 2-methylpyridine, 4-methylpentan-2-one, n-heptane, cyclohexane, methylcyclohexane, 2-(propan-2-yloxy)propane and 2-methoxy-2-methylpropane, and alcohols such as butan-1-ol, butan-2-ol, propan-2-ol, propan-1-ol, 2-methylpropan-1-ol, ethanol and methanol, and mixtures thereof as well as mixtures of the solvents with water.

10. The method according to claim 8, characterized in that the inert solvent is a mixture of ethanol and water.

11. A method for preparing the crystalline modification II of the compound of the formula (I) of claim 1, characterized in that 4-({(2S)-2-[4-{5-chloro-2-[4-(trifluoromethyl)-1H-1,2,3-triazol-1-yl]phenyl}-5-methoxy-2-oxopyridin-1(2H)-yl]butanoyl}-amino)-2-fluorobenzamide acetone having the following formula (III) 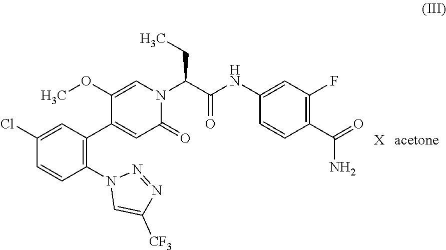
is dried in an oven under reduced pressure, preferable for one day at 50° C. and 10 mbar.

12. 4-({(2S)-2-[4-{3-chloro-2-fluoro-6-[4-(trifluoromethyl)-1H-1,2,3-triazol-1-yl]phenyl}-5-methoxy-2-oxopyridin-1(2H)-yl]propanoyl}amino)-2-fluorobenzamide having the formula (II) 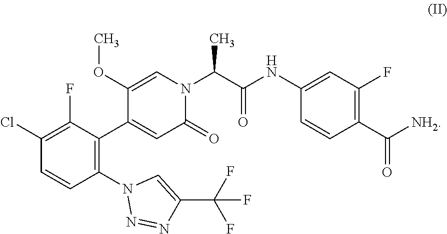 [SMILES: COc1cn([C@@H](C)C(=O)Nc2ccc(C(N)=O)c(F)c2)c(=O)cc1-c1c(-n2cc(C(F)(F)F)nn2)ccc(Cl)c1F]
